(19) United States
(12) Patent Application Publication
(10) Pub. No.: US 2025/0032687 A1
(43) Pub. Date: Jan. 30, 2025

(54) PERITONEAL DIALYSIS SYSTEM AND METHODS

(71) Applicant: Outset Medical, Inc., San Jose, CA (US)

(72) Inventors: James RITSON, San Jose, CA (US); Michael Edward HOGARD, Odessa, FL (US); Gopi K. LINGAM, San Jose, CA (US); Shih-Paul CHEN, Los Altos, CA (US); Dean HU, San Leandro, CA (US)

(21) Appl. No.: 18/593,221

(22) Filed: Mar. 1, 2024

Related U.S. Application Data
(63) Continuation of application No. 18/059,181, filed on Nov. 28, 2022, now Pat. No. 11,951,241.
Application No. 18/059,181 is a continuation of application No. 16/325,664, filed on Feb. 14, 2019, now Pat. No. 11,534,537.
(60) Provisional application No. 62/377,416, filed on Aug. 19, 2016.

(51) Int. Cl. A61M 1/16; A61M 1/14; A61M 1/28; A61M 5/142; A61M 5/145; A61M 5/158; A61M 39/10; C02F 9/00; C02F 103/02

(52) U.S. Cl. CPC A61M 1/1656; A61M 1/153; A61M 1/155; A61M 1/168; A61M 1/1686; A61M 1/287; A61M 5/1452; A61M 5/1454; A61M 5/158; C02F 9/00; A61M 2005/14252; A61M 2005/1426; A61M 2005/14268; A61M 2005/1585; A61M 2039/1066; A61M 2209/04; A61M 2209/045; C02F 2103/026

(57) ABSTRACT

Described herein are systems and methods for performing peritoneal dialysis. According to one aspect, the disclosure provides a sterile interface connection for connecting a water purification system to a disposable source of dialysate concentrates. The sterile interface connection can include a chamber comprising an inlet adapted to be connected to the water purification system on a proximal end and a valve on a distal end. The sterile interface connection can also include spring-loaded needle disposed in the chamber. The spring-loaded needle can move between a retracted configuration in which the spring-loaded needle is fully retracted into the chamber and the valve is closed and sealed, and an extended configuration in which the spring-loaded needle extends through the valve into the disposable source of dialysate concentrates.

### CROSS REFERENCE TO RELATED APPLICATIONS

The present application is a continuation of U.S. patent application Ser. No. 18/059,181, filed Nov. 28, 2022, which is a continuation of U.S. patent application Ser. No. 16/325,664, filed Feb. 14, 2019, titled “PERITONEAL DIALYSIS SYSTEM AND METHODS”, now U.S. Pat. No. 11,534,537, which is a national phase application under 35 USC 371 of International Patent No. PCT/US2017/047791, filed Aug. 21, 2017, titled “PERITONEAL DIALYSIS SYSTEM AND METHODS”, which claims priority under 35 USC 119 to U.S. Provisional Application No. 62/377,416, filed Aug. 19, 2016, titled “PERITONEAL DIALYSIS SYSTEM AND METHODS”, the disclosure of each of which is incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to dialysis systems. More specifically, this disclosure relates to peritoneal dialysis systems that include many features that reduce the need for disposables storage by using on-demand water and making setup and delivery easier for the patient.

### BACKGROUND

There are, at present, hundreds of thousands of patients in the United States with end-stage renal disease. Most of those require dialysis to survive. Many patients receive hemodialysis treatment at a dialysis center, which can place a demanding, restrictive and tiring schedule on a patient. Patients who receive in-center dialysis typically must travel to the center at least three times a week and sit in a chair for 3 to 4 hours each time while toxins and excess fluids are filtered from their blood. After the treatment, the patient must wait for the needle site to stop bleeding and blood pressure to return to normal, which requires even more time taken away from other, more fulfilling activities in their daily lives. Moreover, in-center patients must follow an uncompromising schedule as a typical center treats three to five shifts of patients in the course of a day. As a result, many people who dialyze three times a week complain of feeling exhausted for at least a few hours after a session.

Peritoneal dialysis provides an alternative to hemodialysis in which fluid is introduced into the peritoneum through a permanent tube in the abdomen. Peritoneal dialysis is typically performed in a patient's home while the patient sleeps. The main complication from peritoneal dialysis is infection of the abdomen through the permanent tube.

Traditional peritoneal dialysis requires the batch preparation of fluid that is terminally sterilized before being consumed by the patient. In addition, traditional peritoneal dialysis systems require the patient to make numerous fluid connections. This batch preparation of fluid and numerous setup steps makes treatment in the home difficult.

### SUMMARY

Described herein are apparatus including a sterile interface connection configured to connect a water purification system to a disposable source of dialysate concentrates, the sterile interface connection comprising a chamber comprising an inlet adapted to be connected to the water purification system on a proximal end and a valve on a distal end, a spring-loaded needle disposed in the chamber, a proximal end of the spring-loaded needle being connected to the inlet, wherein the sterile interface connection includes a retracted configuration in which the spring-loaded needle is fully retracted into the chamber and the valve is closed and sealed, the sterile interface connection further including an extended configuration in which the spring-loaded needle extends through the valve into the disposable source of dialysate concentrates.

The spring-loaded needle may further include a lumen extending therethrough and a port disposed at a distal end of the spring-loaded needle. The spring-loaded needle may also be configured to deliver water from the water purification system to the disposable source of dialysate concentrates.

The chamber may further comprise an outlet, the sterile interface connection being configured to, in the retracted position, receive fluid from the water purification system via the inlet to sterilize the chamber and the spring-loaded needle and discard the fluid through the outlet. The fluid may be configured to contact all interior and exterior surfaces of the spring-loaded needle.

Described herein are apparatus including a sterile interface connection of a disposable source of dialysate concentrates, the sterile interface connection being configured to receive a needle of a water purification system to mate the water purification system to the disposable source of dialysate concentrates, the sterile interface connection comprising an alignment feature adapted to guide the needle into the sterile interface connection, a disinfectant plug adjacent to the alignment feature, the disinfectant plug being configured to sterilize the needle as it passes through the disinfectant plug, and a sealing septum adjacent to the disinfectant plug, the scaling septum adapted to form a seal around the needle when the needle punctures the sealing septum.

The interface connection can include a sterile seal disposed over the disinfectant plug, wherein the sterile seal is to be removed from the sterile interface connection prior to needle entry.

Described herein are methods, including a method of connecting a water purification system to a disposable source of dialysate concentrates, comprising receiving a sterile interface connection comprising a chamber, a spring-loaded needle disposed in the chamber, and a valve of the chamber that is closed and sealed, connecting an inlet of the sterile interface connection to the water purification system, extending the spring-loaded needle through the valve into the disposable source of dialysate concentrates.

Any of the methods may further comprise delivering fluid from the water purification system to the disposable source of dialysate concentrates through the sterile interface connection.

Any of the methods may further comprise receiving fluid from the water purification system via an inlet in the sterile interface connection to sterilize the chamber and the spring-loaded needle. Additionally, the methods may include discarding the fluid through an outlet of the sterile interface connection. The fluid can contact all interior and exterior surfaces of the spring-loaded needle.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates one embodiment of a peritoneal dialysis system.

FIG. 2 shows a schematic diagram of the water purification system 102 of FIG. 1.

FIG. 3 shows a schematic diagram of an alternate water purification system that uses de-ionization technology to remove ionic impurities prior to the ultrafilter.

FIG. 4 illustrates a reusable portion of a sterile interface connection that bridges the water purification system to the disposable consumable.

FIGS. 5 and 6 show a disposable portion of the sterile interface connection that bridges the water purification system to the disposable consumable.

FIG. 7 illustrates a concentrate container that contains pre-sterilized dialysate concentrates proportioned to the appropriate dosage for treatment.

FIG. 8 illustrates an automated cycler of a peritoneal dialysis system which feeds the dialysis solution to and from the patient's peritoneum.

FIG. 9 shows an automated cycler that incorporates a dialyzer.

FIG. 10 shows one example of a sterile vessel.

### DETAILED DESCRIPTION

This disclosure describes systems, devices, and methods related to peritoneal dialysis therapy. In some embodiments, the peritoneal dialysis system can be a home dialysis system. Embodiments of the dialysis system can include various features that automate and improve the performance, efficiency, and safety of dialysis therapy.

In some embodiments, a dialysis system is described that can provide acute and chronic peritoneal dialysis therapy to users. The system can include a water purification system configured to prepare water for use in dialysis therapy in real-time using available water sources, and a disposable consumable that interfaces with the water purification system to produce dialysate for dialysis therapy.

In some embodiments, a peritoneal dialysis system can prepare substantially sterile water in real-time for creating dialysis solution. The peritoneal dialysis systems described can also include features that make it easy for a patient to self-administer therapy. For example, a disposable consumable with minimal connection points can be easily installed for automated mixing and movement of fluids. Methods of use are also provided, including real-time dialysis solution monitoring, recirculation of dialysis solutions, and interface connections.

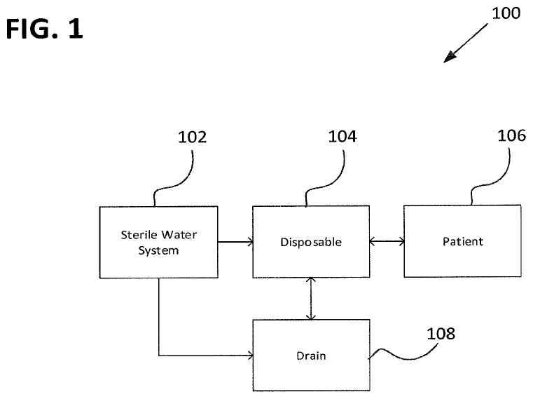

FIG. 1 illustrates one embodiment of a peritoneal dialysis system 100 configured to provide dialysis treatment to a user in either a clinical or non-clinical setting, such as the user's home. The dialysis system 100 can comprise a sterile water system 102 connected to a disposable consumable 104. The disposable consumable 104 can interface with a catheter in the abdomen of the patient 106. Both the sterile water system and the disposable consumable can be connected to a drain 106.

The water purification system 102 can be configured to purify a water source in real-time for dialysis therapy. For example, the water purification system can be connected to a residential water source (e.g., tap water) and prepare pasteurized water in real-time. The pasteurized water can then be used for peritoneal dialysis therapy without the need to heat and cool large batched quantities of water typically associated with water purification methodologies.

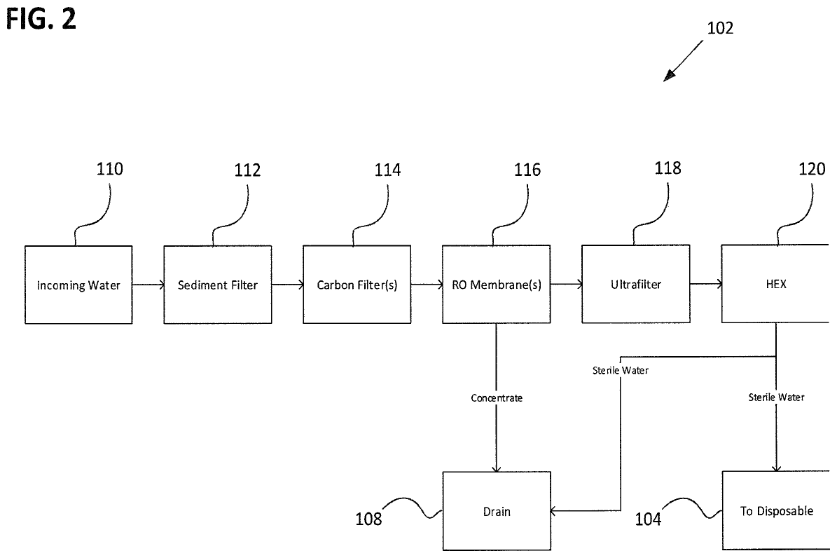

FIG. 2 shows a schematic diagram of the water purification system 102 of FIG. 1. The water purification system can include a source of incoming water 110 (e.g., a tap), one or more sediment filter(s) 112, carbon filter(s) 114, reverse-osmosis (RO) membrane(s) 116, ultrafilter(s) 118, and a heat exchanger (HEX) 120.

The source of incoming water 110 provides supply water to the water purification system, which passes through the sediment filter(s) 112 to filter out particulates in the fluid.

Next, the fluid passes through the carbon filter(s) 114 to filter toxins such as chlorine and chloramine. In some embodiments, there could be multiple carbon filters to ensure that chlorine and chloramine levels are within acceptable range for downstream components.

The fluid then passes through the RO membranes 116 which reject ions in the fluid. A preferred rejection ratio of the RO membranes can be at least 94%. In other embodiments, the rejection ratio can be at least 90%. The permeate water continues toward the ultrafilter 118, while the concentrated ions removed from the fluid by the RO membranes are directed to the drain 108. In some embodiments, there can be a plurality of RO membranes to maximize permeate water retention.

As the permeate water traverses across the ultrafilter(s) 118, any bacteria or endotoxins adhere to the ultrafilter(s). In one specific embodiment, the ultrafilter can be a polysulfone membrane with a pore size of at least 0.01 microns. In some embodiments, there can be several ultrafilters in series to ensure water purity. In other embodiments, the ultrafilter(s) may be located at other points in the fluid path, including before the RO membranes.

Finally, the permeate water is passed through a pasteurization system or HEX 120, that uses elevated pressures and temperatures to denatured and inactivate all the endotoxins in the permeate water. It has been shown that elevating endotoxin to a temperature above 200 C at a pressure of 240 PSI can provide more than a log 2 reduction in endotoxin level.

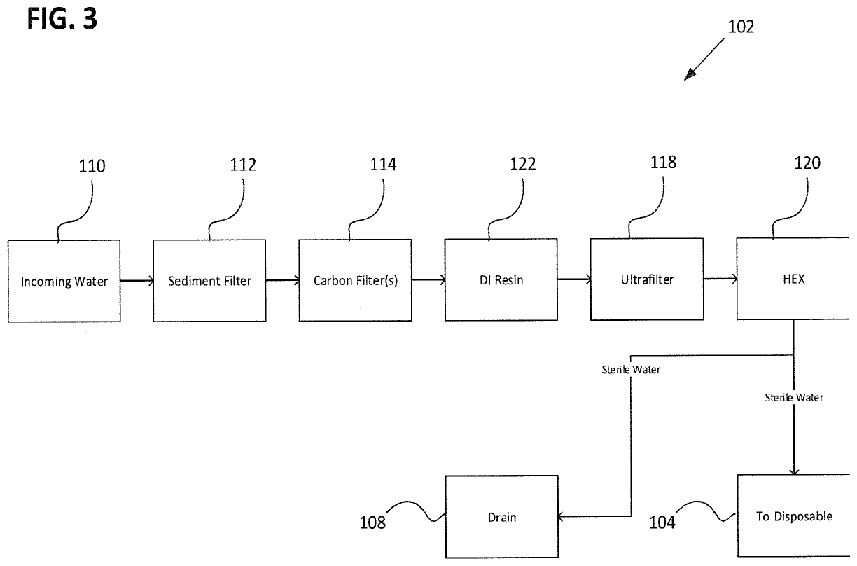

FIG. 3 shows a schematic diagram of an alternate water purification system that uses de-ionization technology to remove ionic impurities prior to the ultrafilter. Instead of using RO membranes before the ultrafilter as in the embodiment of FIG. 2, the water purification system 102 of FIG. 3 incorporates one or more de-ionizing resin(s) 122 to capture undesired ions and output pure water.

Once sterile water device reaches temperature and pressure conditions in which endotoxins and bacteria are actively denatured and inactivated, the water can be directed from the water purification system to the disposable consumable. The disposable consumable can include a number of features, described below.

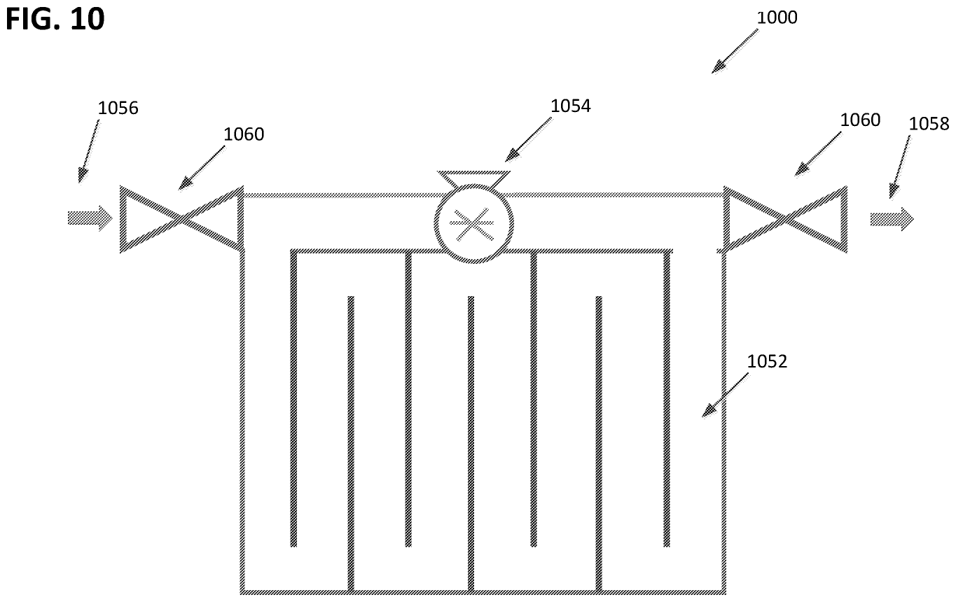

In another embodiment, the sterile water coming from the water purification system can be stored into a sterile vessel. The vessel can be self-disinfectable from water produced by the heat exchanger, and may contain actuator elements to circulate fluid within itself to prevent dead volumes from occurring. In some embodiments, the sterile vessel may be one open volume, wherein contained fluid may be periodically or continuously agitated by one or a plurality of rotary mixers. In one embodiment, as shown in FIG. 10, a sterile vessel 1000 may comprise a substantially non-branched fluid path 1052 arranged in a serpentine configuration, or other configuration which allows for a long linear path length in a small volume. One or more circulation pumps 1054 disposed within the fluid paths cause the fluid to circulate. The inlet 1056 and outlet 1058 of the fluid path may be controllably opened by valves 1060 to allow water in and out of the fluid path. Sterile water emerging from the vessel can then be mixed with the contents of the concentrate container in a homogenous fashion, and be raised to patient temperature.

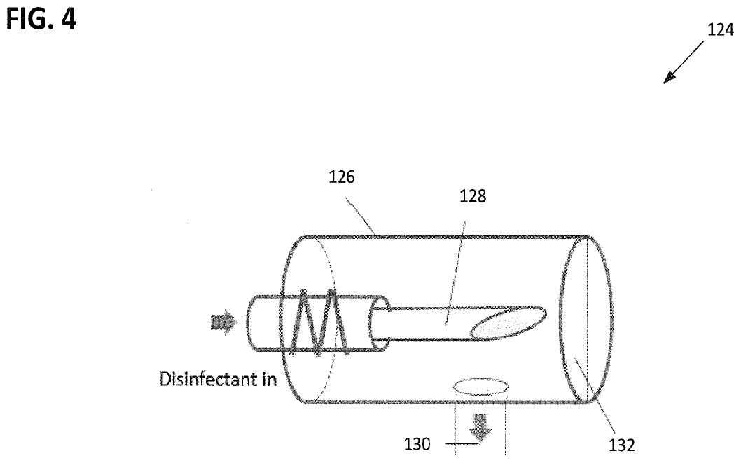

FIGS. 4-6 illustrate a reusable portion of a sterile interface connection that bridges the water purification system to the disposable consumable. The sterile interface connection can include a reusable connection 124 on the water purification system, and a disposable connection 125 on the disposable consumable. The chances for contamination in the peritoneal dialysis system are limited by having only one interface between the disposable consumable and the water purification system.

Referring to FIG. 4, the reusable connection 124 can include a chamber 126, a spring loaded needle 128, a chamber outlet 130, and a self-opening and self-scaling door 132. The reusable connection 124 can include a retracted configuration and an extended configuration. In the retracted configuration, the spring loaded needle 128 can be retracted into the chamber 126 through the door 132 to limit exposure to the ambient environment. In this retracted position, the water purification system can to rinse the interface with disinfectant, sterile water, or a combination of the two. In the extended configuration, the spring loaded needle 128 can extend through the door 132 to puncture or be inserted into the disposable consumable.

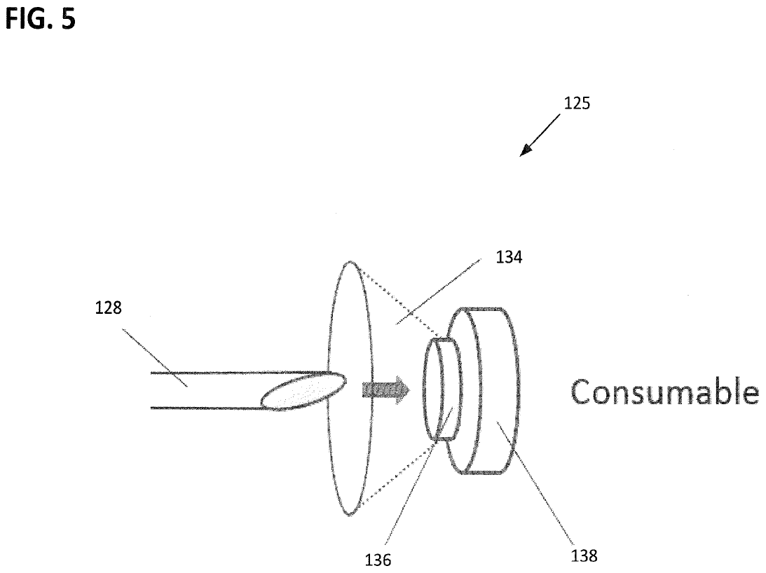

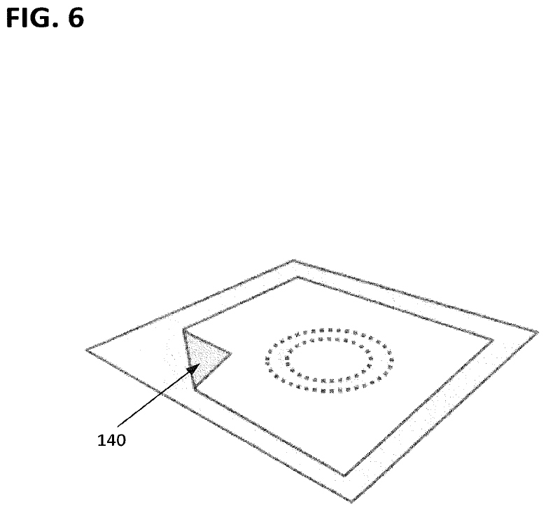

Referring to FIGS. 5-6, the disposable connection 125 of the sterile interface connection can have a puncture point that is be pre-sterilized until active use. The puncture point can be manually exposed or automatically exposed prior to active use. In one embodiment, referring to FIG. 5, the puncture point of the disposable connection 125 may include an alignment feature 134 configured to guide needle 128 into the puncture point. In the illustrated embodiment, the puncture point can include a disinfectant plug 136 and a sealing septum 138. The sealing septum can comprise an elastomer or similar material. Upon needle puncture, the scaling septum 138 can form a seal around the needle 128, thereby creating a non-leaking connection between the sterile water system and the disposable.

In another embodiment, referring to FIG. 6, the puncture point can be covered with a sterile seal 140 that is removable by the user prior to active use. Peeling the removable seal may be manual or automatic in nature, but sterility of the puncture point can be maintained.

Systems and methods are also provided for the creation, disposal, and recycling of dialysate solution prior to, during, and after treatment.

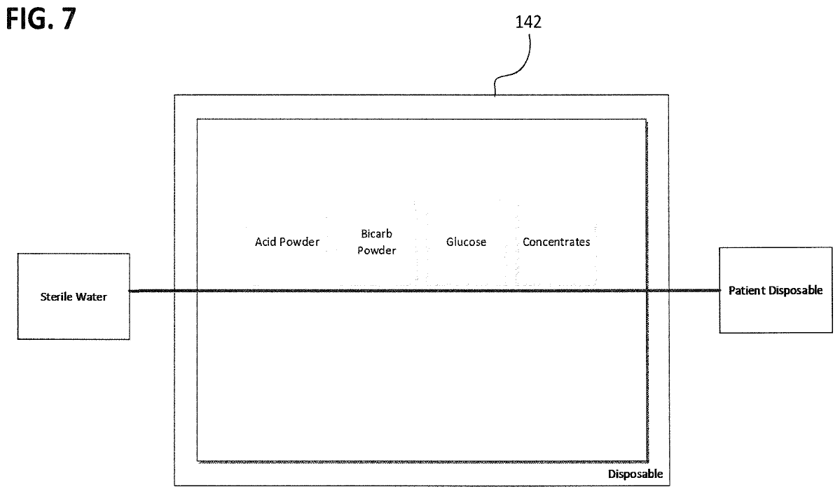

Referring to FIG. 7, the disposable consumable can include a concentrate container 142 that contains pre-sterilized dialysate concentrates proportioned to the appropriate dosage for treatment. In one embodiment, the concentrate container can include acid powder, bicarb powder, glucose, and other concentrates. In some embodiments, these concentrates can be used to produce the oncotic or osmotic gradient required for conducting peritoneal dialysis. In some embodiments, these concentrates may contain high molecular weight substances such as polysaccharide, polyamides or polypeptides in order to minimize transfer of these substances into the patient. Over the course of a typical overnight peritoneal dialysis session, several infusion/drain cycles may be performed, wherein the peritoneal dialysis fluid is infused into the patient's peritoneum, allowed to dwell, and drained. Because there a plurality of concentrates available, the composition of the dialysis fluid infused for each cycle may be varied, for example, to achieve different clinical goals, such as increased fluid removal, minimization of glucose retention, or maintenance of nutritional compounds that may be lost during the drain cycle. In some embodiments, the disposable consumable may have electrodes built into it, connectable to an external sensor for real-time dialysis fluid monitoring.

Sterile water coming from the water purification system can be mixed with the contents of the concentrate container in a homogenous fashion, and be raised to patient temperature. In some embodiments, the disposable consumable comprises two thin, thermally conductive, flexible sheets welded together in a pattern to produce tortuous flow paths that promote mixing of fluids. The disposable consumable may further comprise tubing sections, or other features which can be acted upon by non fluid-contacting pumps on the durable portion of the invention. Temperature, conductivity or other properties of the fluid within the disposable consumable may be sensed or measured by non-contact sensors connected to the durable portion of the invention. Heat may also be applied to the fluid in the disposable consumable through at least one side of the thin, thermally conductive flexible sheets.

Once fully mixed, the dialysis solution could then be routed to a patient with an automated cycler, thereby enabling real-time peritoneal dialysis solution generation and delivery. The benefit of real-time peritoneal dialysis solution generation during automated peritoneal dialysis is such that the patient does not have to make multiple connections from multiple sources, thus increasing the risk of peritonitis. Furthermore, the use of batch prepared dialysis solution can be burdensome for the patient, as bags containing liters of solution are relatively heavy for the typical dialysis patient. This system minimizes intense physical activity for the patient prior to initiating a treatment.

In another embodiment, the fully mixed dialysis solution could then be routed to a sterile dialysis fluid vessel via an automated cycler. This would enable batching of the peritoneal dialysis solution prior to delivery.

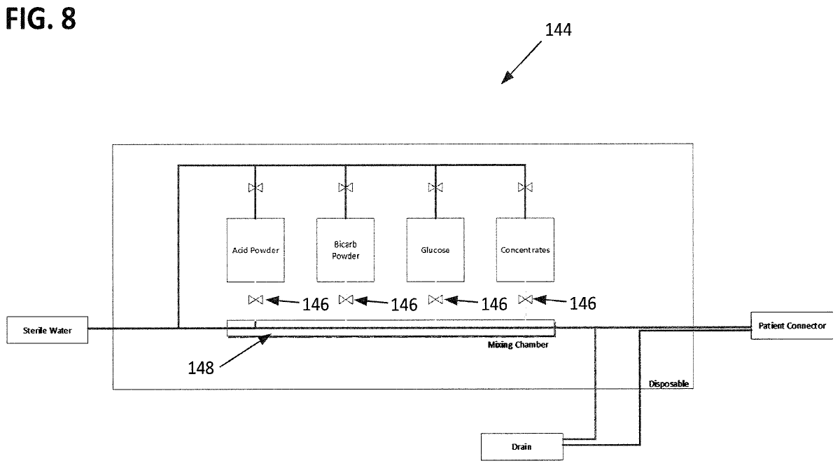

FIG. 8 illustrates an automated cycler 144 of the disposable consumable of a peritoneal dialysis system, which feeds the dialysis solution to and from the patient's peritoneum. The automated cycler 144 can contain all of the non-contact pumps and sensors that interact with the dialysate solution to deliver the solution to the patient. When the spring loaded needle of the reusable connection is connected to the disposable connection, as described above, the internal concentrate lines 146 can be opened. Pumps of the automated cycler, such as peristaltic pumps, can be used to pump supersaturated mixtures of each concentrate into a mixing chamber 148. The fluid within the mixing chamber can then be elevated to the patient temperature.

As proportioning of the dialysis solution is critical, a conductivity sensor will mate to the electrical leads of the disposable consumable to allow the cycler to monitor the quality of the dialysis fluid in real-time. Should the dialysis fluid not meet the settings prescribed on the prescription, the cycler will be able to route the fluid to drain until proportioning is corrected. The conductivity sensor can be placed, for example, at an outlet of the mixing chamber.

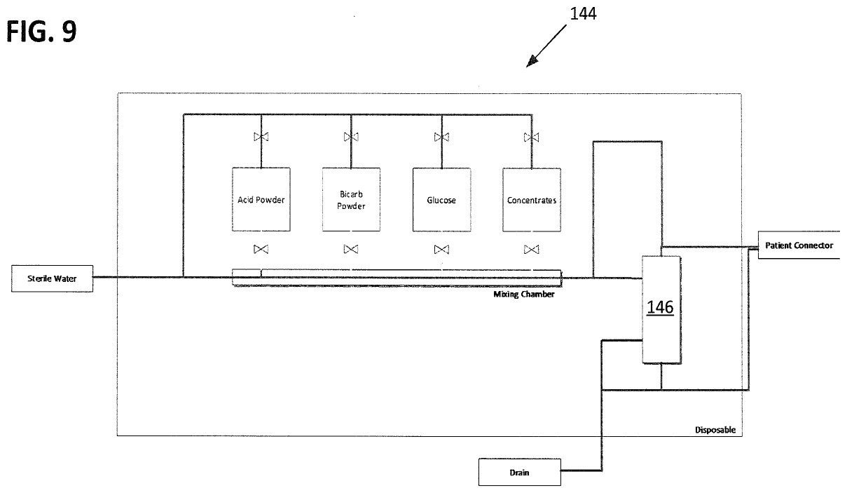

As extreme nutrient loss is extremely prevalent in peritoneal dialysis, it may be desirable to recycle the patient effluent dialysis solution as to prevent critical proteins from leaving the body. Referring to FIG. 9, the automated cycler 144 can incorporate a dialyzer 146 that allows for the diffusion or convection of waste from one fluid source to another. In this embodiment, the dialysis solution side of the dialyzer can be constantly be replenished with new dialysis solution flowing in one direction, while the patient effluent solution side of the dialyzer can move fluid in the opposite direction as to maximize waste clearances. The dialyzer may also be bypassed, in events for filling or draining the peritoneum.

As peritonitis is a common, yet unexpected, outcome of peritoneal dialysis, early detection of peritonitis symptoms can be detected through the constant monitoring of drain line. In particular, a turbidity meter to monitor patient effluent can be placed in or around the drain line to monitor the change in discoloration of the fluid. Once detected, the automated cycler can alarm the user of the onset of peritonitis and take appropriate action.

1. (canceled)

2. A sterile interface connection of a disposable source of dialysate concentrates, the sterile interface connection being configured to receive a needle of a water purification system to mate the water purification system to the disposable source of dialysate concentrates, the sterile interface connection comprising:
an alignment feature adapted to guide the needle into the sterile interface connection;
a disinfectant plug adjacent to the alignment feature, the disinfectant plug being configured to sterilize the needle as it passes through the disinfectant plug; and
a sealing septum adjacent to the disinfectant plug, the sealing septum adapted to form a seal around the needle when the needle punctures the sealing septum.

3. The sterile interface connection of claim 2, further comprising a sterile seal disposed over the disinfectant plug, wherein the sterile seal is to be removed from the sterile interface connection prior to needle entry.

4. The sterile interface connection of claim 2, wherein the sealing septum comprises an elastomer.

5. The sterile interface connection of claim 2, wherein the alignment feature is funnel shaped.

6. The sterile interface connection of claim 2, wherein the sealing septum is configured to create a non-leaking connection between the water purification system and the disposable source of dialysate.
